(19) United States
(12) Patent Application Publication
(10) Pub. No.: US 2020/0384156 A1
(43) Pub. Date: Dec. 10, 2020

(54) INJECTABLE MESH

(71) Applicant: LifeCell Corporation, Madison, NJ (US)

(72) Inventors: Hui Xu, Plainsboro, NJ (US); Eric Stec, Washington, NJ (US); Ming F. Pomerleau, Califon, NJ (US)

(21) Appl. No.: 16/894,171

(22) Filed: Jun. 5, 2020

Related U.S. Application Data
(60) Provisional application No. 62/858,731, filed on Jun. 7, 2019.

(51) Int. Cl. A61L 27/36; A61L 27/22; C12N 9/10; A61L 27/44

(52) U.S. Cl. CPC A61L 27/3641; A61L 27/362; A61L 27/225; A61L 27/44; A61L 27/222; C12N 9/1044; A61L 27/227

(57) ABSTRACT

The present application relates to injectable tissue products and their production and use thereof. The tissue products include a group of acellular tissue matrix particles, a bioadhesive, and a biocompatible polymer. The tissue products may be used to treat hernia defects.

This application claims priority under 35 U.S.C. § 119 to U.S. Provisional Application No. 62/858,731, filed Jun. 7, 2019, the entire contents of which is incorporated herein by reference.

The present disclosure relates to tissue products, including an injectable mixture of acellular tissue matrix particles, bioadhesives, and polymers.

Various tissue-derived products are used to regenerate, repair, or otherwise treat diseased or damaged tissues and organs. Such products can include intact tissue grafts or acellular or reconstituted acellular tissues (e.g., acellular tissue matrices from skin, intestine, or other tissues, with or without cell seeding). Such products can also include hybrid or composite materials, e.g., materials including a synthetic component mixed with materials derived from tissue.

Tissue products, including acellular tissue matrices (ATMs), can be used for a variety of load bearing or regenerative applications. In many situations, implantation of a tissue product requires surgical access to a tissue site, e.g., via an incision, and a method of fixing the tissue product to the tissue site. To prevent or reduce surgical complications related to fixation, reduce surgery time, and improve overall quality of care, it may be desirable to produce tissue products that are injectable into a tissue site but become set, fixed, or harden in situ to form a load-bearing structure that does not require additional fixation. Such products could be used, for example, to treat anatomic defects such as hernias or fistulas.

Accordingly, the present disclosure provides devices and methods that provide injectable tissue products including ATM particles, bioadhesives, and biocompatible polymers. The devices and methods can provide one or more of improved mechanical reinforcement, conformity to specific patient anatomy, adherence without external fixation, enhanced tissue regeneration, reduced likelihood of foreign body response, chronic inflammation, and subsequent chronic pain. The devices and methods can also promote tissue regeneration.

### SUMMARY

In one embodiment, an injectable treatment composition is provided. The composition can include a group of acellular tissue matrix particles, a bioadhesive, and a biocompatible polymer. In some embodiments, the composition solidifies in vivo. In some embodiments, the polymer is configured to provide mechanical reinforcement to a hernia defect. In some embodiments, the composition adheres to surrounding tissue. In some embodiments, the acellular tissue matrix particles comprise a slurry. In some embodiments, the acellular tissue matrix particles comprise acellular dermal tissue matrix. In some embodiments, the acellular tissue matrix particles comprise acellular muscle tissue matrix. In some embodiments, the acellular tissue matrix particles comprise porcine acellular tissue matrix. In some embodiments, the bioadhesive is at least one of: transglutaminase, fibrin glue, in situ polymerized polyurethane, and albumin glutaraldehyde. In some embodiments, the biocompatible polymer is at least one of: silk fibroin, chitosan, polylactic-co-glycolic acid (PLGA), and polydioxanone (PDS). In some embodiments, the biocompatible polymer is a gelatin.

In another embodiment, a method of treating an anatomic site with a tissue product is provided. The method can include selecting a defect in an anatomic site and injecting a treatment composition into the anatomic site. The composition includes a group of acellular tissue matrix particles, a bioadhesive, and a biocompatible polymer. In some embodiments, the composition solidifies in the anatomic site. In some embodiments, the composition solidifies in the form of a polymer mesh. In some embodiments, the anatomic site is a hernia defect. In some embodiments, the acellular tissue matrix particles comprise a slurry. In some embodiments, the composition adheres to surrounding tissue. In some embodiments, the acellular tissue matrix particles comprise acellular dermal tissue matrix. In some embodiments, the acellular tissue matrix particles comprise acellular muscle tissue matrix. In some embodiments, the acellular tissue matrix particles comprise porcine acellular tissue matrix. In some embodiments, the bioadhesive is at least one of: transglutaminase, fibrin glue, in situ polymerized polyurethane, and albumin glutaraldehyde. In some embodiments, the biocompatible polymer is at least one of: silk fibroin, chitosan, polylactic-co-glycolic acid (PLGA), and polydioxanone (PDS). In some embodiments, the biocompatible polymer is a gelatin.

In another embodiment, a method of producing an injectable treatment composition is provided. The method can include selecting an acellular tissue matrix. The method can include mechanically processing the acellular tissue matrix to produce acellular tissue matrix particles. The method can include adding a bioadhesive to the acellular tissue matrix particles. The method can include adding a biocompatible polymer to the acellular tissue matrix particles. In some embodiments, the acellular tissue matrix comprises acellular dermal tissue matrix. In some embodiments, the acellular tissue matrix comprises acellular muscle tissue matrix. In some embodiments, the acellular tissue matrix comprises porcine acellular tissue matrix. In some embodiments, the bioadhesive is at least one of: transglutaminase, fibrin glue, in situ polymerized polyurethane, and albumin glutaraldehyde. In some embodiments, the biocompatible polymer is at least one of: silk fibroin, chitosan, polylactic-co-glycolic acid (PLGA), and polydioxanone (PDS). In some embodiments, the biocompatible polymer is a gelatin. In some embodiments, the biocompatible polymer is added in a solution. In some embodiments, the solution comprises the polymer dissolved in an aqueous buffer at a concentration ranging from 20% to 30% polymer. In some embodiments, the biocompatible polymer is added in the form of particulate.

### DESCRIPTION OF THE DRAWINGS

FIG. 1 is a graph depicting the relationship of gelation kinetics to transglutaminase concentration, according to Example 1.

FIG. 2 is a bar graph depicting the tensile max load of an exemplary tissue product as a function of gelatin concentration, according to Example 1.

FIG. 3 are hematoxylin & eosin stained section of an exemplary product implanted in a rat subcutaneous model and explanted at four weeks.

FIG. 4 are hematoxylin & eosin stained section of an exemplary product implanted in a rat subcutaneous model and explanted at twelve weeks.

### DESCRIPTION OF CERTAIN EXEMPLARY EMBODIMENTS

Reference will now be made in detail to certain exemplary embodiments according to the present disclosure, certain examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Any range described herein will be understood to include the endpoints and all values between the endpoints.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

Various human and animal tissues can be used to produce products for treating patients. For example, various tissue products for regeneration, repair, augmentation, reinforcement, and/or treatment of human tissues that have been damaged or lost due to various diseases and/or structural damage (e.g., from trauma, surgery, atrophy, and/or long-term wear and degeneration) have been produced. Such products can include, for example, acellular tissue matrices, tissue allografts or xenografts, and/or reconstituted tissues (i.e., at least partially decellularized tissues that have been seeded with cells to produce viable materials).

A variety of tissue products have been produced for treating soft and hard tissue defects. For example, ALLODERM® and STRATTICE® (ALLERGAN®, NJ) are two dermal acellular tissue matrices made from human and porcine dermis, respectively. Although such materials are very useful for treating certain types of conditions, it may be desirable to modify the tissue matrices or other tissue products to alter the surface mechanical properties, to improve resistance to wear or damage, to prevent development of adhesions with surrounding tissues, or to reduce friction when the tissue products are in contact with other materials such as body tissue.

Source tissues are used to create acellular tissue matrices used to form various moldable tissue matrix products and compositions. The acellular tissue matrix may originate from a human or an animal tissue matrix. Suitable tissue sources for an acellular tissue matrix may include allograft, autograft, or xenograft tissues. Human tissue may be obtained from cadavers. Additionally, human tissue may be obtained from live donors; e.g. autologous tissue.

Some examples of non-human tissue sources which may be used for xenograft tissue matrices include pig, cow, sheep, or other animals from domestic or wild sources and/or any other suitable mammalian or non-mammalian xenograft tissue source. In some exemplary embodiments, the acellular tissue matrix may originate from a source dermal matrix taken from an animal, such as a pig. In one exemplary embodiment, the source dermal matrix may comprise one or more layers of skin that have been removed from an animal.

If porcine or other animal sources are used, the tissue may be further treated to remove antigenic components, such as 1,3-alpha-galactose moieties, which are present in pigs and other mammals, but not humans or certain other primates. In some embodiments, the tissue is obtained from animals that have been genetically modified to lack expression of antigenic moieties, such as 1,3-alpha-galactose, for example. See Xu, Hui, et al., “A Porcine-Derived Acellular Dermal Scaffold that Supports Soft Tissue Regeneration: Removal of Terminal Galactose-α-(1,3)-Galactose and Retention of Matrix Structure,” Tissue Engineering, Vol. 15,1-13 (2009), which is hereby incorporated by reference in its entirety.

Acellular tissue matrices can provide a suitable tissue scaffold to allow cell ingrowth and tissue regeneration. Starting materials for forming an injectable tissue product can include an acellular dermal matrix (“ADM”), in some embodiments. In some embodiments, the ADM is a porcine acellular dermal matrix (“pADM”). In some embodiments, the ADM is a human ADM. Other sources of ADM could be used, as previously mentioned. The starting ADM material may comprise substantially non-cross-linked collagen to allow infiltration with host cells, including fibroblasts and vascular elements. Regardless, some degree of collagen cross-linking may result from processing the ADM.

The acellular tissue matrix particles are formed by subjecting the source tissue matrix to mechanical and/or chemical processing steps. Mechanical processing generally removes undesired tissues and reduces the source tissue into smaller particles. For example, a sheet of acellular tissue matrix may be shredded into particles. Mechanical processing may further include grinding, grating, freeze-drying, fracturing, or other processes to break apart tissue. The source tissue matrix may be checked for fatty tissue and cut to remove the tissue and/or to prevent tangling of tissue matrix pieces. The source tissue matrix may be frozen and thawed prior to mechanical processing. In some embodiments, the acellular tissue matrix particles are formed into a slurry.

In some embodiments, the tissue matrix particles are sorted by size. In an exemplary embodiments, sequentially sized wire screens filter the particles into groups of particles within a similar size range.

A bioadhesive may be added to the ATM particles. Exemplary bioadhesives include, but are not limited to, microbial transglutaminase, fibrin glue, in situ polymerized polyurethane, albumin glutaraldehyde, laccase, tyrosinase, or lysyl oxidase. Non-enzymatic based crosslinking agents such as carbodiimide, bissulfosuccinimidyl suberate, genipin, and 1, 4-butanediol diglycidyl ether can also or alternatively be used. Discussion of non-enzymatic based crosslinking agents as bioadhesives can be found in MATHEIS, GUNTER, and JOHN R. WHITAKER. “A review: enzymatic cross-linking of proteins applicable to foods.” Journal of Food Biochemistry 11.4 (1987): 309-327, which is herein incorporated by reference.

Transglutaminases are enzymes expressed in bacteria, plants, and animals that catalyze the binding of gamma-carboxyamide groups of glutamine residues with amino groups of lysine residues or other primary amino groups. Transglutaminases are used in the food industry for binding and improving the physical properties of protein rich foods such as meat, yogurt, and tofu. Transglutaminases are also currently being explored for use in the medical device industry as hydrogels and sealants. See Aberle, T. et al., “Cell-type Specific Four Component Hydrogel,” PLoS ONE 9(1): e86740 (January 2004).

For example, the transglutaminase can be provided in a solution or formed into a solution from a stored form (e.g., a dry powder or other suitable storage form). The solution can include any suitable buffer such as phosphate buffered saline or other biologically compatible buffer material that will maintain or support enzymatic activity and will not damage the enzyme or tissue product.

A variety of transglutaminases can be used including any that are biologically compatible, can be implanted in a patient, and have sufficient activity to provide desired catalytic results within a desired time frame. Transglutaminases are known and can include microbial, plant, animal, or recombinantly produced enzymes. Depending on the specific enzyme used, modifications such as addition of cofactors, control of pH, or control of temperature or other environmental conditions may be needed to allow appropriate enzymatic activity. Microbial transglutaminases can be effective because they may not require the presence of metal ions, but any suitable transglutaminase may be used in the methods described below.

The tissue products can further include a biocompatible polymer. In some embodiments, the biocompatible polymer is a gelatin. In further embodiments, the gelatin is produced from cold water fish or pigs. In some embodiments, the gelatin has a gel strength (bloom number) of 300.

In some embodiments, the polymer is provided in a solution. In other embodiments, the polymer is provided in particulate form. In some embodiments, the polymer is dissolved in an aqueous buffer such as phosphate buffered saline, sodium acetate, or sodium citrate. In further embodiments, the polymer is dissolved in an aqueous buffer at a concentration ranging from 20% to 30% polymer.

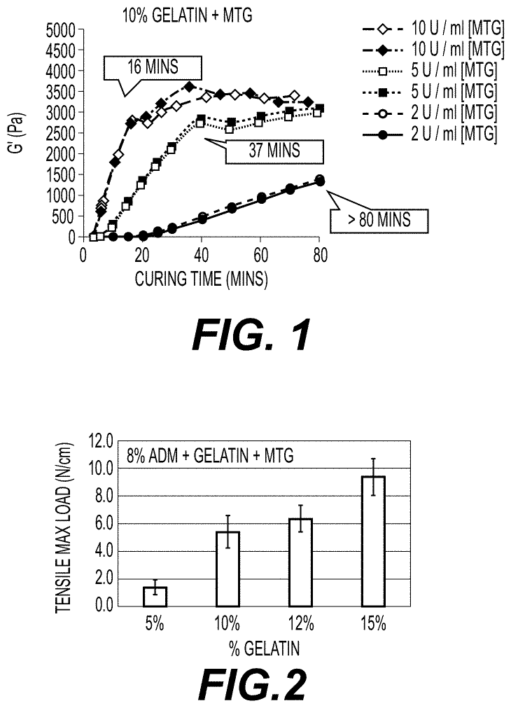

In some embodiments, the tissue product is a composition that is injectable through a cannula or needle and becomes set, solidified, or stabilized once in contact with tissue in situ. The product may comprise a mixture including a group of acellular dermal tissue matrix particles, microbial transglutaminase, and gelatin. The mixture of the acellular dermal tissue matrix particles, microbial transglutaminase, and gelatin facilitates an enzymatic crosslinking reaction between glutamine and lysine residues on the gelatin and matrix proteins to form in situ gelation. The tissue product will also adhere to surrounding tissue. The gelation kinetics of the tissue product may be adjusted by varying the transglutaminase concentration as depicted in FIG. 1. An appropriate curing time will allow surgeons sufficient time to work with the tissue product materials, while minimizing the time needed to apply the tissue product to a tissue site.

The mechanical strength of the tissue product is dependent on multiple factors. For example, increasing the concentration of certain components of the tissue product will increase the degree of crosslinking in the tissue product and enhance the mechanical strength of the tissue product. For example, as shown in FIG. 2, a material cured using 15% (w/w) gelatin, 7.5 U/ml microbial transglutaminase, and 8% (w/w) acellular dermal tissue matrix particles achieved a tensile maximum load of 9.4±1.3 N/cm (maximum stress of 248 kPa). The morphology and particle size of the acellular tissue matrix particles also play a role. A more fibrous collagen matrix will lead to a more interconnected network and improve the mechanical strength of the resultant tissue product. Shear adhesive strength of an exemplary tissue product was tested using a burst test model with 15% defect area. The burst strength of a tissue product cured using 10% (w/w) gelatin, 5 U/ml microbial transglutaminase, and 8% (w/w) acellular dermal tissue matrix particles was 13.3±2.3 N.

The tissue products and their methods of production can be used for the treatment of a variety of conditions. For example, the tissue product may be injected into a tissue site containing a hernia defect. In some embodiments, the tissue product may be used for inguinal hernia repair, umbilical hernia repair, fistula repair, a minimally invasive facelift procedure, filling a void (i.e., a lumpectomy), site specific augmentation (such as the temples), or pelvic prolapse repair. For example, the tissue product may be used to treat a parastomal hernia or a hiatal hernia.

After injection, the tissue product hardens to form an implant within the tissue site. In some embodiments, the tissue product hardens to form a mesh product. For example, the biocompatible polymer may harden into the form of a mesh. Such mesh products may be capable of treating a hernia site, for example. In some embodiments, the polymer mesh provides mechanical reinforcement to the hernia defect.

In some embodiments, the tissue product is injected after an incision is made at a treatment site. The tissue product may then be injected within the area of the incision. In such embodiments, the incision may be closed after injection, either before or after the tissue product hardens.

### EXAMPLE 1

Three different formulations of injectable tissue products with varying gelatin and microbial transglutaminase (MTG) concentrations were tested in a competent rat subcutaneous study. The compositions of test groups are summarized in Table 1.

| | Gelatin | MTG | ADM | Max Load |
|---|---|---|---|---|
| Group | (% w/w) | (U/ml) | (% w/w) | (N/cm) |
| 1 | 15% | NA | 8% | NA |
| 2 | 5% | 5 | 8% | 1.4 ± 0.6 |
| 3 | 10% | 5 | 8% | 6.8 ± 0.7 |
| 4 | 15% | 7.5 | 8% | 9.4 ± 1.3 |

The gelatin prepared was fish gelatin dissolved in a sodium acetate buffer at a pH of 6. The microbial transglutaminase was dissolved in sodium citrate at a pH of 6 but can be alternatively dissolved in water.

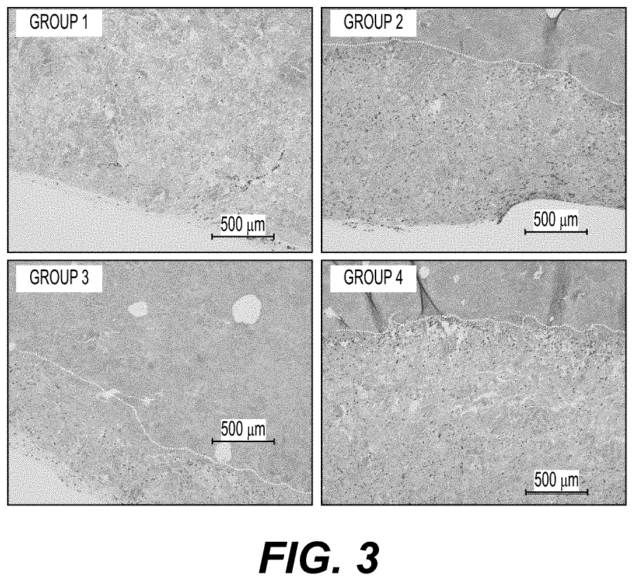

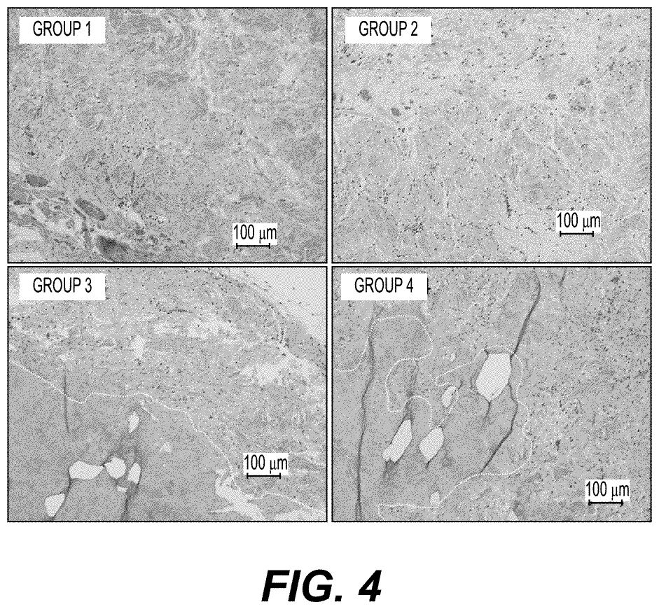

Acellular dermal tissue matrix particles and the microbial transglutaminase were mixed. The resulting mixture was then mixed with the gelatin. The resulting tissue products were injected subcutaneously into the rats to induce in situ curing. At four weeks after injection, cell infiltration and revascularization were found in the periphery of cured tissue products, and inflammation were observed to be low to medium (See FIG. 3). At twelve weeks after injection, cell infiltration penetrated deeper into the cured tissue products, occupying 50% or more of the cured tissue product when examined cross-sectionally using H&E staining (See FIG. 4). Revascularization was achieved in the areas where cells infiltrated. At both time points, integration and adherence to surrounding tissue were achieved, with no signs of seroma or hematoma. Explant weight of the test groups reduced by 30-60% from four to twelve weeks, while the explant weight of the control group remained unchanged. The explant weight loss is likely due to enzymatic degradation and remodeling.

The above description and embodiments are exemplary only and should not be construed as limiting the intent and scope of the invention.

1. An injectable treatment composition comprising:
a group of acellular tissue matrix particles;
a bioadhesive; and
a biocompatible polymer.

2. The composition of claim 1, wherein the composition solidifies in vivo.

3. The composition of claim 1, wherein the polymer is configured to provide mechanical reinforcement to a hernia defect.

4. The composition of claim 1, wherein the composition adheres to surrounding tissue.

5. The composition of claim 1, wherein the acellular tissue matrix particles comprise a slurry.

6. The composition of claim 1, wherein the acellular tissue matrix particles comprise acellular dermal tissue matrix.

7. The composition of claim 1, wherein the acellular tissue matrix particles comprise acellular muscle tissue matrix.

8. The composition of claim 1, wherein the bioadhesive is at least one of:
transglutaminase, fibrin glue, in situ polymerized polyurethane, and albumin glutaraldehyde.

9. The composition of claim 1, wherein the biocompatible polymer is at least one of:
silk fibroin, chitosan, polylactic-co-glycolic acid (PLGA), and polydioxanone (PDS).

10. The composition of claim 1, wherein the biocompatible polymer is a gelatin.

11. A method of treating a defect, comprising:
selecting a defect in an anatomic site;
injecting a treatment composition into the anatomic site, wherein the composition comprises:
a group of acellular tissue matrix particles;
a bioadhesive; and
a biocompatible polymer.

12. The method of claim 11, wherein the composition solidifies in the anatomic site.

13. The method of claim 12, wherein the anatomic site is a hernia defect.

14. The method of claim 13, wherein the composition is configured to provide mechanical reinforcement to the hernia defect.

15. The method of claim 12, wherein the composition solidifies in the form of a polymer mesh.

16. The method of claim 11, wherein the composition adheres to surrounding tissue.

17. The method of claim 11, wherein the acellular tissue matrix particles comprise a slurry.

18. The method of claim 11, wherein the acellular tissue matrix particles comprise acellular dermal tissue matrix.

19. The method of claim 11, wherein the acellular tissue matrix particles comprise acellular muscle tissue matrix.

20. The method of claim 11, wherein the bioadhesive is at least one of:
transglutaminase, fibrin glue, in situ polymerized polyurethane, and albumin glutaraldehyde.

21. The method of claim 11, wherein the biocompatible polymer is at least one of:
silk fibroin, chitosan, polylactic-co-glycolic acid (PLGA), and polydioxanone (PDS).

22. The method of claim 11, wherein the biocompatible polymer is a gelatin.
